(19) United States
(12) Patent Application Publication
(10) Pub. No.: US 2021/0093998 A1
(43) Pub. Date: Apr. 1, 2021

(54) DEVICE AND METHOD FOR THE SEQUESTRATION OF ATMOSPHERIC CARBON DIOXIDE

(71) Applicant: ONE STIFTUNGS GMBH, Heidelberg (DE)

(72) Inventor: Jan-Heiner KÜPPER, Kleinkoschen (DE)

(73) Assignee: ONE STIFTUNGS GMBH, Heidelberg (DE)

(21) Appl. No.: 16/761,313

(22) PCT Filed: Nov. 5, 2018
(86) PCT No.: PCT/EP2018/080134

§ 371 Date: May 4, 2020

(30) Foreign Application Priority Data
Nov. 4, 2017 (EP) 17200037.4

(51) Int. Cl. B01D 53/62; B01D 53/04; B01D 53/053; B01D 53/85; B01D 19/00; C12M 1/00; C12N 1/12

(52) U.S. Cl. CPC B01D 53/62; B01D 53/0462; B01D 53/053; B01D 53/85; B01D 19/0057; B01D 2259/802; C12M 43/00; C12N 1/12; B01D 2257/504; B01D 2251/95; C12M 21/02

(57) ABSTRACT

The invention relates to a device and to a method for sequestering atmospheric carbon dioxide using at least one air capture module in conjunction with a bioreactor equipped with an autotrophic microorganisms.

The invention relates to a device and to a method for sequestering atmospheric carbon dioxide using an air capture module in functional conjunction with a bioreactor equipped with autotrophic microorganisms.

The need to quantitatively sequester carbon dioxide (CO₂) from the atmosphere is viewed as a global problem. In addition to significantly reducing the use of fossil fuels, a direct sequestration of CO₂ from the atmosphere is considered to be necessary to be able to achieve the worldwide climate targets. These consist in a maximum permissible temperature increase of less than 2° C. compared to when record-keeping began. Other measures, such as geoengineering, for example by iron fertilization of the ocean or the introduction of sulfur compounds into the atmosphere so as to enhance the reflection of solar radiation, are rated as very risky, with ecological consequences.

According to the findings of climate researchers, an average global temperature rise of more than 2° C. results in irreversible disruptions of the climate systems. Another global problem is the decarbonization of industry, which often accompanies the phase-out of the use of fossil fuels and energy sources. This means that carbon sources other than fossil sources have to be found for chemical processes.

Technology describes the direct sequestration of carbon dioxide from the atmosphere using bioenergy with carbon capture and storage (BECCS). This involves using cultivated crops for energy purposes (biomass and gas-fired power plants) and storing the arising CO₂ in geological strata. BECCS, however, has the following drawbacks: 1.) CO₂ injection into geological strata, which is associated with risks and only possible in few regions of the earth. 2.) Competition with agriculture since the high land requirement for BECCS results in a shortage of cultivation space for food production.

The use of photobioreactors, which contain autotrophically growing microorganisms and produce biomass, is considered to be a promising option of carbon dioxide sequestration. For example, it is easy to use microalgae. This biomass has a variety of uses, such as 1.) biogas generation for energy production, 2.) recovery of carbon compounds for the chemical industry, 3.) biofuels, and 4.) food additives, which can be contained especially in algae, 5.) other valuable substances such as pharmaceutically acting substances and cosmetics, 6.) organic fertilizer made of biomass (biofertilizer).

In the prior art, WO 1998/045409 A1 and EP 2 568 038 A1 describe laminar photobioreactors for the production of microalgae, wherein the following problems are discussed:

a.) A suitable microorganism has to be used, which is easy and inexpensive to cultivate and has a high biomass production.

b.) A continuous CO₂ supply must be ensured since the atmospheric CO₂ concentration in the amount of 400 ppm (0.04%) does not allow optimal growth of microalgae, for example. It was found that, at optimal CO₂ concentrations, microalgae create biomass approximately 10 to 50 times more efficiently than crops. The technical teaching describes that microalgae such as Chlorella, Scenedesmus, Spirulina, Nannochloropsis, Nostoc and Chlorococcus are able to grow very well in the range of 1 to 20% CO₂ (that is approximately 25 to 500 times higher than in the atmosphere), and have an accordingly high biomass productivity (see also Appl. Biochem. Biotechnology, 2016 179:1248-1261 and the literature cited therein). Previously, the problem was solved by using chemically pure CO₂ (technical CO₂). Of course, this does not solve the problem of carbon sequestration since this CO₂ is obtained in a highly energy-consuming process as a by-product in the chemical industry. A variety of working groups have already attempted to use alternatives in the form of waste gas flows from power plants. Even though this would allow the CO₂ arising during the combustion of fossil energy sources to be sequestered, it would not ensure a direct removal of CO₂ from the atmosphere. Moreover, it is known that waste gas flows from power plants contain impurities such as sulfur, nitrogen oxides, carbon monoxide and heavy metals, which can drastically inhibit the growth of microorganisms. Removing harmful impurities from these waste gas flows is a very cost-intensive process. In contrast, a direct introduction of atmospheric air into photobioreactors would have the drawbacks that, first, too little CO₂ is present for optimal growth and, secondly, that algae predators such as protozoa and zooplankton may be present on small dust particles in the air. These organisms subsist on algae and can thus heavily disrupt the bioreactor operation.

c.) For microalgae to grow optimally, it is necessary that the oxygen that develops during the light reaction is removed since it can have a toxic effect, and moreover also triggers the process of photorespiration, wherein CO₂ is formed again.

d.) Another problem is that an efficient bioreactor should allow a continuous operation, that is, the supply of nutrient solution and removal of biomass take place constantly, without having to stop the reactor. Moreover, a bioreactor should be configured so flexibly that different types of microalgae, and even prokaryotic chemolithotrophic CO₂ fixers, can be cultivated.

e.) The photobioreactor has to maintain optimal growth conditions of the microorganism, such as temperature, pH value, nutrients and the like.

The prior art, however, does not describe a suitable device and method for sequestering atmospheric carbon dioxide using a bioreactor, and in particular a photobioreactor.

It is therefore the object of the invention to provide a suitable device or a method for sequestering atmospheric carbon dioxide by producing biomass.

To achieve this object, the invention thus relates to a device for sequestering atmospheric carbon dioxide, wherein at least one module comprising a capture unit binds atmospheric carbon dioxide by way of an adsorber material and, after treatment by way of heat or a vacuum, the atmospheric carbon dioxide is kept available, and the module is connected to at least one bioreactor, wherein atmospheric carbon dioxide is continuously supplied to autotrophic microorganisms in at least one bioreactor.

In another preferred embodiment, the invention relates to a device for sequestering atmospheric carbon dioxide, wherein at least one module comprising a capture unit binds atmospheric carbon dioxide by way of an adsorber material and, after treatment by way of heat or a vacuum, the atmospheric carbon dioxide is kept available in a container, in particular a pressurized container, wherein atmospheric carbon dioxide is continuously supplied to autotrophic microorganisms in at least one bioreactor.

In another embodiment, the invention relates to a device for sequestering atmospheric carbon dioxide, comprising a module comprising a capture unit, wherein atmospheric carbon dioxide is bound by way of an adsorber material and, after treatment by way of heat or a vacuum, the atmospheric carbon dioxide is kept available in a pressurized container, and at least one bioreactor containing autotrophic microorganisms.

A pressure reducer can be assigned to such a pressurized container, so that a continuous CO₂ stream can be provided, if necessary using measuring and control technology.

In another preferred embodiment, atmospheric carbon dioxide can be supplied to autotrophic microorganisms in at least one bioreactor together with air. Ratios of 5:95 vol. % CO₂/air, and in particular from 1:99 vol. % CO₂/air to 10:90 vol. % CO₂/air, are preferred.

To achieve this object, the invention thus likewise relates to a method for sequestering atmospheric carbon dioxide, wherein at least one module comprising a capture unit binds atmospheric carbon dioxide by way of an adsorber material and, after treatment by way of heat or a vacuum, the atmospheric carbon dioxide is kept available, and the module is connected to at least one bioreactor, wherein atmospheric carbon dioxide is continuously supplied to autotrophic microorganisms in at least one bioreactor.

The prior art describes the sequestration of CO₂ from industrial waste gases by way of a bioreactor, which, however, is entirely different, since such waste gases are of a different quality and, air contains other harmful substances and has an insufficient CO₂ concentration.

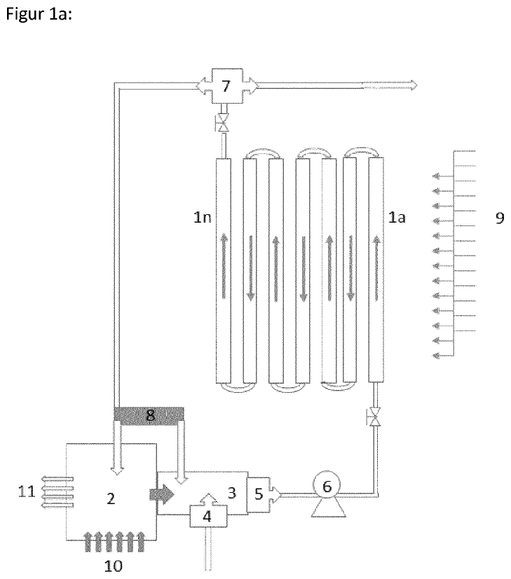

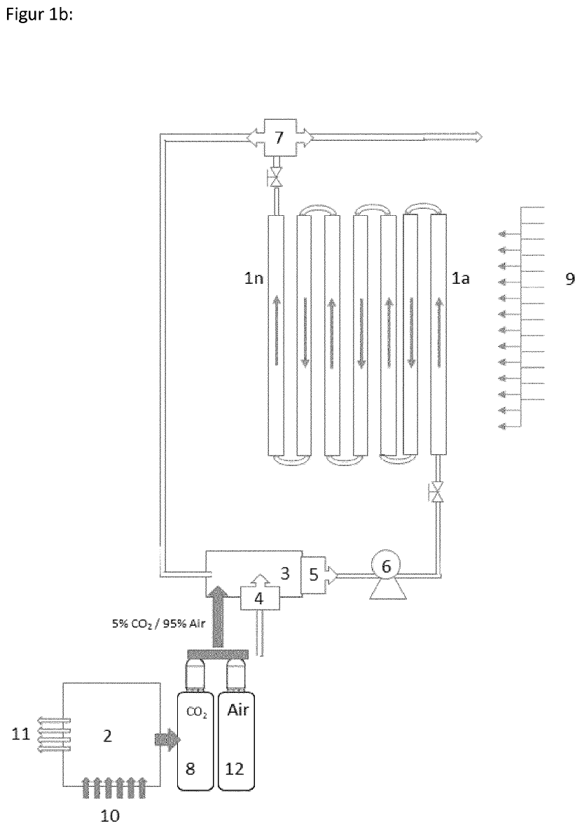

In a preferred embodiment, the device according to the invention includes such features according to FIG. 1a or FIG. 1b, whereby the above-described problems can be solved completely for the first time.

Preferably, bioreactor modules that run parallel and are connected to one another are used (1a-1n, FIGS. 1a, 1b). These are fed a nutrient solution including the autotrophic microorganism to be cultivated, preferably microalgae of the genus chlorella, Scenedesmus, Spirulina, Nannochloropsis, Nostoc or Chlorococcus (3, FIGS. 1a, 1b). Chemically pure CO₂ is introduced into the nutrient solution, preferably together with air, wherein the CO₂ preferably stems from a connected air capture module (carbon dioxide recovery installation) (2, FIGS. 1a, 1b). In particular, the aforementioned algae exhibit favorable growth rates in the device according to the invention, including the method according to the invention that is carried out.

The company Climeworks in Switzerland (http://www.climeworks.com/) produces functional air capture modules, for example, which can be connected to the bioreactor in accordance with the invention. Atmospheric CO₂ is bound by way of these air capture modules (10, FIGS. 1a, 1b) and can subsequently be released again by way of heating at approximately 100° C. In contrast, atmospheric oxygen or nitrogen is not bound, but is returned into the atmosphere (11, FIGS. 1a, 1b). By combining the air capture module with a bioreactor, it is achieved for the first time that atmospheric CO₂ is pre-concentrated in a form that is optimal for microorganisms, without additional interfering components, such as harmful substances or algae predators, being present. The latter are efficiently destroyed by the heating process for CO₂ release.

A measuring and control unit (5, FIGS. 1a, 1b) measures critical parameters such as the CO₂ concentration, pH value, algae biomass per unit of volume. Thereafter, the solution is transferred into the bioreactor via a system pump (6, FIGS. 1a, 1b). In the case of photobioreactors, illumination takes place (9, FIGS. 1a, 1b). As a result of the translucency of the material, the preferred algae according to the invention, serving as the microorganism, are able to carry out photosynthesis. The optimal CO₂ concentration, which can be flexibly set by way of the air capture module, causes considerable reproduction in the reactor modules. Algae biomass can, on the one hand, be given off continuously via a central measuring and control unit (7, FIGS. 1a, 1b) and processed by way of common methods.

On the other hand, this is preferably a continuous bioreactor, which can operate in a circuit. The algae are conducted across a vapor-liquid separator (also: gas-liquid separator) (8, FIG. 1a). The principle of gas separation from a photobioreactor operated with microalgae is known. For example, the algae can be conducted through a chamber containing a semipermeable membrane, by which the gases (O₂/CO₂) present in the liquid are removed by way of diffusion. Another technical solution is the use of a mechanical, vortex-driven gas separator (Fasoulas et al., University of Stuttgart, status report on the 2nd preliminary result within the scope of the project 50 JR 1104 “Regenerative Lebenserhaltungssysteme fur die Raumfahrt mit synergetisch integrierten Photobioreaktoren and Brennstoffzellen (Regenerative life-sustaining systems for the aerospace industry with synergetically integrated photobioreactors and fuel cells)” funded by the DLR space agency in the time period, 2014). The gas (oxygen and unconsumed CO₂) is returned into the air capture module via the separator (2, FIGS. 1a, 1b). In the process the O₂ escapes, wherein the CO₂ is bound again and conducted into the circuit. This advantageously solves the problem of the continuous removal of O₂. The algae are conducted from the vapor-liquid separator into the central cultivation tank again (3, FIGS. 1a, 1b). Here, the CO₂ concentration can now be set to the optimal value again, and nutrient solution can be supplied from outside (4, FIGS. 1a, 1b).

The invention thus relates to such a device according to the invention which additionally comprises a gas-liquid separator, so that a continuous circulatory process can advantageously be achieved, and arising oxygen can be removed.

In another preferred embodiment, 5 to 50% of the culture medium or nutrient solution is replaced within a day. The device comprises a measuring unit (7, FIG. 1a), for example, which opens a faucet at a defined biomass concentration (for example, 1 g/liter, measured by way of the optical density (OD650ₙₘ) of the medium) so as to conduct a defined proportion of the culture medium into a collection vessel. At the same time, the missing and fresh culture volume (4, FIG. 1a) is supplied again.

The installation can likewise be operated with chemo(litho)autotrophic bacteria, such as Archaea bacteria, which likewise receive CO₂ via the air capture module. A light reaction is not required, but an energy source in the form of H₂ (molecular hydrogen) is.

Within the meaning of the present invention, the expression “autotrophic microorganisms” thus encompasses those microorganisms that utilize light as an energy source (photoautotrophic microorganisms) or a chemical energy source (such as hydrogen) (chemoautotrophic microorganisms). Autotrophic microorganisms are able to carry out carbon dioxide fixation and create biomass in this way.

Within the meaning of the present invention, a “bioreactor” can synonymously be referred to as a fermenter and is used to cultivate the autotrophic microorganisms for producing biomass, wherein according to the invention a continuous operation of the bioreactor is preferred. A person skilled in the art is able to set appropriate operating parameters, for example, for algae, among other things microorganisms, by way of a measuring and control system (temperature, pH value of the culture solution and the like), and to provide culture media. A photobioreactor as described in WO 1998/045409 A1 and EP 2 568 038 A1 is furthermore preferred.

Monosaccharides and/or polysaccharides, and more particularly glucose, can be added in a concentration of 0.3 to 10 g/L culture medium as another advantageous carbon source in a culture medium.

Within the meaning of the present invention, an “air capture module” is able to capture atmospheric CO₂ by way of a capture unit according to the invention, having a large surface, wherein the CO₂ is chemically or physically bound by way of an adsorber or filter, such as sodium hydroxide, amines or cellulose. By way of heating (for example, to 50 to 120 degrees Celsius) and/or a vacuum, the CO₂ can be brought into the gas phase again by the reusable capture unit or filter, so as to be conducted in a concentrated form into a bioreactor in accordance with the invention, preferably by way of a first container, and in particular a pressurized container. An “air capture module” thus relates to a first device, wherein a capture unit (or container) chemically or physically binds atmospheric CO₂ using an adsorber material and keeps it available in a container, in particular a pressurized container, after the treatment by way of heat and/or a vacuum.

The company Climeworks AG, Switzerland, specializes in the air capture technology. The chemical fixation capacity per module is approximately 35 kg/CO₂ per hour, and can be increased to a scale of tons/hour by utilizing multiple modules. This allows the provision of large amounts of CO₂ for the gasification of the autotrophic microorganisms for CO₂ fixation in a bioreactor, likewise in a continuous operation.

Such an air capture module is used to recover carbon dioxide from the ambient air and, if needed, likewise provides condensation water from the ambient air for further material use. Preferably, a carbon dioxide recovery installation is selected which initially binds carbon dioxide from the air current using an adsorption operation and, thereafter, releases the carbon dioxide for further use by way of a temperature and/or vacuum process.

The aforementioned device can likewise be described as a method and can also encompass the use of this device for sequestering atmospheric carbon dioxide.

The biomass that is obtained and produced can be used for the usual applications, such as the production of biofuel, chemical substances, energy use and the like (supra).

The following examples are provided to describe the invention, however without limiting the subject matter of the invention.

### EXAMPLE 1

Adsorption Operation:

Ambient air is taken in by a container (capture unit) filled with adsorber material using a fan. The ambient air usually contains 0.04 vol. % carbon dioxide and, depending on climate, a certain amount of water vapor. The carbon dioxide accumulates to a high degree at the surface of the adsorber material, which contains sodium hydroxide, amines or cellulose. Moreover, water accumulates at the surface of the adsorption material, wherein usually at least 2 moles of water per 1 mole of carbon dioxide, however, at least 1 mole per 1 mole of carbon dioxide, is adsorbed.

Regeneration is required when the surface of the adsorber material is saturated or enriched with carbon dioxide. This can take place by way of heat and/or a vacuum, wherein the physically or chemically bound CO₂ (or carbonate) is converted into the form of a gas again and is collected in a container and, if necessary, buffered and, if necessary, compressed. The temporary buffering of the carbon dioxide in a short-term storage device and in a long-term storage device connected in parallel thereto can take place at increased pressure. After cooling, the adsorber material can be reused.

### EXAMPLE 2

Flat Plate Photobioreactor Example:

A flat plate photobioreactor from the company IGV (Potsdam, Germany) is used. It is composed of planar chambers that are connected to tubes and vertically positioned in series. The chambers are rectangular and have an edge length of 1 m and a depth of 2 cm. This results in a volume of 20 liters each. Five chambers connected in series result in a total volume of 100 liters. The flow is driven by way of the system pump, as shown in FIG. 1 (6). The CO₂ is conducted across the air capture module “Demonstrator” from Climeworks (Switzerland) into a gas buffer module (Climeworks, Switzerland), which temporarily buffers up to 2 m³ of the concentrated CO₂ from the installation until use. The CO₂ uptake and release and the current concentration are recorded in the gas buffer module by way of CO₂ sensor technology. Together with sterile air, it is conducted from this module into the flat plate photobioreactor. The combination (2) of the air capture module and the gas buffer module is schematically illustrated in FIG. 1a. This air capture module “Demonstrator” is able to make up to 8 kg atmospheric CO₂ from the atmosphere per day available for the installation. The CO₂ taken up at the surface of the air capture module is released again at 100° C. by heating and conducted into the gas buffer module. By way of this module, gaseous CO₂ is conducted into the bioreactor together with air in a metered manner. In this way, a CO₂ concentration is set in the nutrient medium as a function of desired conditions. For example, the photobioreactor is gasified with a mixture of 5% CO₂ and air. The composition of the gas (for example, 5% v/v CO₂, 95% v/v air) is controlled and regulated externally by way of a gas mixing station (BBi biotech, Berlin).

The photobioreactor is exposed to light by way of LEDs from the company Valoya Oy (Helsinki, Finland). The LEDs used are the BX90 series (88 W) having the spectra AP67 and NS1. This covers the majority of the visible light spectrum. Each plate module of the photobioreactor is exposed separately to LED lighting. The arrangement is advantageously selected in such a way that an input photon flux density of approximately 110 μmol/m²s is achieved, which is excellently suited for spirulina, for example.

### EXAMPLE 3

Production of Algae Biomass using a Flat Plate Photobioreactor:

Sterile culture medium having the following composition is added into the installation (Aiba, S. and Ogawa T. 1976, Assessment of Growth Yield of a Blue-green Alga, Spirulina platensis, in Axenic and Continuous Culture. Journal of General Microbiology 102, 179-182):

NaHCO₃ (4.05×10⁻² M) , Na₂CO₃ (9.50×10⁻³ M) , K₂HPO₄ (7.17×10⁻⁴ M) , NaNO₃ (7.35×10⁻³ M) , K₂SO₄ (1.43×10⁻³ M) , NaCl (4.27×10⁻³ M) , MgSO₄×7H₂O (4.15×10⁻⁴ M) , CaCl₂×2H₂O (9.01×10⁻⁵ M), FeSO₄×7 H₂O (1.64×10⁻⁵ M), EDTA=Titriplex III (0.04 g/L)+2.5 ml/L micro nutrient medium (2.2 mg/L ZnSO₄×7 H₂O, 25 mg/L MnSO₄×4 H₂O, 28 mg/L H₃BO₃, 2 mg/L Co [NO₃]2×6 H₂O, 0.21 mg/L Na₂ MoO₄×2H₂O, 0.79 mg/L CuSO₄×5 H₂O)+1 ml/L Vitamin B12 (1.5 g/L) . The pH value is 9.3.

Initially, a sterile starter culture (1 L) is inoculated with Spirulina platensis (Culture Collection of Algae Gottingen, SAG) in the above-described nutrient solution in a shake flask (shake frequency of 100 to 120 rpm) and cultivated in the batch for 3 to 4 days. The photon flux density (PFD) is set to 100 to 150 μmol/m²s. The gasification is carried out by way of a cotton stopper and diffusion.

The flat plate photobioreactor is inoculated with this starter culture, and the entire system (see FIG. 1) is put into operation. It is gasified with a mixture of 5% CO₂/air. The medium is preferably moved by way of a system pump, or the medium can also be circulated by way of a membrane-assisted so-called air-lift technique. The temperature of the nutrient medium in the reactor is preferably 30° C.

The installation is designed so as to be operable in a batch process, that is, the biomass is only harvested once at the end of the experiment. In this case, the bioreactor is operated for 5 to 8 days. The highest productivity, however, is preferably achieved during continuous or semi-continuous operation. A defined proportion of the reactor volume is replaced with fresh culture medium or nutrient medium in the process (see devices 4 and 7 in FIG. 1a). The highest productivity is achieved when 30% of the nutrient medium is replaced every day. In the batch process, the productivity is, on average, 500 to 800 mg algae biomass/liter/day. By continuously replacing the nutrient medium (30% per day), a productivity of 1.5 g algae biomass/liter/day is achieved.

### EXAMPLE 4

Algae biomass using open pond bioreactor (Appl Microbiol Biotechnol (2007) 74:1163-1174)):

Instead of the flat plate photobioreactor, an open system is used, which has a volume of 500 L. The nutrient medium (see above) is continuously circulated using a flow rate of 0.2 to 0.5 m s⁻¹ by way of electrically operated bucket wheel-like paddles. The open pond system is operated in a batch process or in a semi-continuous process. After inoculation with 10 liters of spirulina starter culture (see above), the cultivation is carried out in a batch process up to 7 days. In the semi-continuous process, a certain proportion (for example 10%) of the medium in which the microalgae have multiplied is harvested every day, and replaced with new medium. The open pond system is illuminated in a closed space from above using LEDs of the BX180 series (Valoya, Finland). The open pond system is gasified with a 2.5% CO₂/air mixture. The CO₂ is provided by way of an air capture module. The room temperature is 24° C. After seven days, the biomass is harvested or the bioreactor is run on a semi-continuous basis. The concentration of the biomass is approximately 5 g/L.

### EXAMPLE 5

Example of carbon sequestration by way of humus formation: One of the following microalgae capable of nitrogen fixation is inoculated in the closed photobioreactor or in the open pond system with CO₂ supply (mixture of 2.5% CO₂ and air): Nostoc, Anabaena, Aulosira, Tolypothrix, Nodularia, Cylindrospermum, Scytonema, Aphanothece, Calothrix, Anabaenopsis, Mastigocladus, Fischerella, Stigonema, Haplosiphon, Chlorogloeopsis, Camptylonema, Gloeotrichia, Nostochopsis, Rivularia, Schytonematopsis, Westiella, Westiellopsis, Wollea, Plectonema, Chlorogloea. Nostoc muscorum is well-suited for the open pond system and grows in liquid medium in a manner similar to spirulina. Nostoc muscorum is cultivated for 14 days and then harvested as a batch. As an alternative, a semi-continuous cultivation is carried out, wherein every day approximately 10% of the resultant biomass is harvested, and the withdrawn medium is replaced with fresh culture medium. During the cultivation phase, atmospheric nitrogen is fixed by the algae. The algae biomass is dried. The batch process results in a yield of 700 mg biomass/L.

The dry biomass is pressed to form granules, which are distributed in the soil as biofertilizer. This algae biomass is largely composed of carbon (>50%), which stems from the CO₂ fixation in the case of autotrophic growth. The inoculation of a suitable soil substrate with Nostoc also results in an improvement in the supply of nitrogen. The biomass has a ratio of carbon to nitrogen of 10 to 15:1.

The biofertilizer made of algae biomass improves the growth of plants, such as trees, whereby further CO₂ sequestration is enabled.

### EXPLANATION REGARDING OF THE FIGURES

Legend for FIG. 1a:

1: bioreactor modules that run parallel and are connected to one another, 2: air capture module (optionally including gas buffer module), 3: central cultivation tank, 4: nutrient solution from outside, 5: measuring and control unit for CO₂, pH value, temperature, 6: (system) pump, 7: measuring unit for biomass concentration and control unit for deliberate delivery of culture medium, 8: vapor-liquid separator for separating gas and liquid, 9: illumination in the case of photobioreactor, 10: entry and binding of atmospheric CO₂, 11: exit of atmospheric oxygen or nitrogen.

Legend for FIG. 1b:

1: bioreactor modules that run parallel and are connected to one another, 2: air capture module, 3: central cultivation tank, 4: nutrient solution from outside, 5: measuring and control unit for CO₂, pH value, temperature, 6: (system) pump, 7: measuring unit for biomass concentration and control unit for deliberate delivery of culture medium, 8: CO₂ pressurized container, 9: illumination in the case of photobioreactor, 10: entry and binding of atmospheric CO₂, 11: exit of atmospheric oxygen or nitrogen, 12: compressed air, together with CO₂ a constant ratio of 5% CO₂ and 95% air is conducted by way of a gas mixing station into the bioreactor.

1. A device for sequestering atmospheric carbon dioxide, the device comprising at least one module comprising a capture unit binding atmospheric carbon dioxide by way of an adsorber material and, after treatment by way of heat or a vacuum, the atmospheric carbon dioxide being kept available, and the module being connected to at least one bioreactor, wherein the atmospheric carbon dioxide is continuously supplied to autotrophic microorganisms in at least one bioreactor.

2. The device for sequestering atmospheric carbon dioxide according to claim 1, wherein the atmospheric carbon dioxide is kept available in a container.

3. A device for sequestering atmospheric carbon dioxide, the device comprising
a module comprising a capture unit, wherein atmospheric carbon dioxide is bound by way of an adsorber material and, after treatment by way of heat or a vacuum, the atmospheric carbon dioxide is kept available in a pressurized container, and
at least one bioreactor containing autotrophic microorganisms.

4. A device for sequestering atmospheric carbon dioxide according to claim 1, further comprising at least one gas-liquid separator.

5. A device for sequestering atmospheric carbon dioxide according to claim 1, wherein at least one bioreactor is a photobioreactor or an open pond bioreactor.

6. A device for sequestering atmospheric carbon dioxide according to claim 1, wherein at least one module is an air capture module.

7. A device for sequestering atmospheric carbon dioxide according to claim 1 wherein the autotrophic microorganisms are photoautotrophic microorganisms or chemoautotrophic microorganisms, in particular Archaea bacteria, algae, micro algae, Scenedesmus, Spirulina, Nannochloropsis, Nostoc or Chlorococcus.

8. A device for sequestering atmospheric carbon dioxide according to claim 1, wherein atmospheric carbon dioxide is supplied to autotrophic microorganisms in at least one bioreactor together with air.

9. A device for sequestering atmospheric carbon dioxide according to claim 1, wherein 5 to 50% of the culture medium is replaced.

10. A method for sequestering atmospheric carbon dioxide, at least one module comprising a capture unit binding atmospheric carbon dioxide by way of an adsorber material and, after treatment by way of heat or a vacuum, the atmospheric carbon dioxide being kept available, and the module being connected to at least one bioreactor, wherein atmospheric carbon dioxide is continuously supplied to autotrophic microorganisms in at least one bioreactor.

11. The method for sequestering atmospheric carbon dioxide according to claim 10, wherein a continuous operation of the bioreactor is carried out.

12. A method for sequestering atmospheric carbon dioxide from the ambient air, the method comprising utilizing the device according to claim 1.

13. The device according to claim 2, wherein the container is a pressurized container.

14. The device for sequestering atmospheric carbon dioxide according to claim 7, wherein the algae are of the genus Chlorella.

15. The device for sequestering atmospheric carbon dioxide according to claim 8, wherein the ratio of carbon dioxide to air is from 1:99 vol. % CO₂/air to 10:90 vol. % CO₂/air.
